(19) United States
(12) Patent Application Publication
(10) Pub. No.: US 2014/0364828 A1
(43) Pub. Date: Dec. 11, 2014

(54) INFUSION TRACKER AND RELATED METHODS

(71) Applicant: PRO-IV MEDICAL LTD., Kfar Vitkin (IL)

(72) Inventors: Jacob Nusbacher, Ra'anana (IL); Michal Devir, Kfar Vitkin (IL); Pierre Sharvit, Moshav Gan Yoshia (IL); Gershon Goldenberg, Carcur (IL)

(73) Assignee: PRO-IV MEDICAL LTD., Kfar Vitkin (IL)

(21) Appl. No.: 14/293,035

(22) Filed: Jun. 2, 2014

Related U.S. Application Data
(60) Provisional application No. 61/831,153, filed on Jun. 5, 2013.

(51) Int. Cl. A61M 5/168; G06F 19/00

(52) U.S. Cl. CPC A61M 5/1689; A61M 5/16813; A61M 5/16877; G06F 19/3462; G06F 19/3406; G06F 19/3468; A61M 2205/3576; A61M 2205/52; A61M 2205/50; A61M 2205/6009
USPC 604/500; 604/250

(57) ABSTRACT

An infusion tracker comprising a base and a cover configured to be assembled onto the base. The infusion tracker further comprises a memory tag, attached to one of the base and the cover and configured to store data and operable to wirelessly release stored data in a format readable by a processor-controlled device or to activate an application software. The infusion tracker is further configured to enclose a portion of an infusion line between two ends of the portion, the portion being enclosed substantially between the base and the cover.

### FIELD OF THE INVENTION

The invention, in some embodiments, relates to the field of controlling or authenticating or registering or tracking infusion, and more particularly, but not exclusively, to an infusion tracker configured to assist in controlling or authenticating or registering or tracking infusion, and to related methods.

### BACKGROUND OF THE INVENTION

Intravenous administration of medications or blood transfusion is one of the most common medical therapeutic endeavors. It has been estimated that in US hospitals alone there may be as many as 1 billion intravenous infusions annually. The transfusion of blood products amounts to about an additional 15 million in the US. In addition, intravenous drugs are also administered in outpatient clinics and home care treatments, both hospital-affiliated and free standing, in nursing homes and in physicians' offices. Finally there is an exciting growing industry of home infusions which alleviates the health care system from costly and sometimes hazardous hospitalizations. At home care treatments there is no confidence if the patient follows exactly the physician instructions and prescription. Thus, considerable activity associated with intravenous administration of medications or blood transfusion is carried out in locations or under constraints that preclude or diminish direct control and/or authentication of details associated with such activity.

### SUMMARY OF THE INVENTION

As discussed above, patients receiving intravenous infusions at a distance are in a risk of receiving the wrong medication, or receiving their medications at the wrong time, in the wrong order, or not at all. The current invention creates an electronic monitoring system so that caregivers know in real time what is happening with all their patients who are supposed to receive intravenous medications. In some embodiments the system may alert caregivers when there is non-compliance with doctor's orders and can communicate directly with caregivers to assure appropriate and safe treatment.

According to an aspect of some embodiments there is provided an IV administration set with a new compliance activator. The activator has two parts, one is the base unit including a special firmly groove to hold a standard infusion line, and the second part is the activator cover holding a memory tag like NFC or RFID or similar tags in a special location containing all the necessary details regarding the treatment, the patient and the caregiver. The activator unit is located on the infusion line in any place by a one way connector, the activator is placed on the line in the pharmacy during the bag filling, and according the type of medication filled in the bag. By scanning the memory tag by an HHD, smartphone or another suited device with a dedicated application all the treatment details will be sent in real time mode to the backend software by wireless communication, comparing with the physician order entered before treatment should start. If even one of the compared details is wrong the system sends a hazard message to all point of care locations including the caregiver, the physician and the nurse station.

According to an aspect of some embodiments there is provided an infusion tracker comprising a base and a cover configured to be assembled onto the base. The infusion tracker further comprises a memory tag, attached to one of the base and the cover and configured to store data and operable to wirelessly release stored data in a format readable by a processor-controlled device or to activate an application software.

The infusion tracker has at least a first state, and a second assembled state in which the cover is assembled onto the base. The infusion tracker may be moved from the first state to the second assembled state by manually pressing the cover towards the base, and the infusion tracker may not be moved from the second assembled state to the first state by pressing the cover towards the base nor by releasing a pressure therefrom.

The infusion tracker, in the second assembled state is further configured to enclose a portion of an infusion line between two ends of the portion, the portion being enclosed substantially between the base and the cover. Fluid communication substantially exists between the two ends of the portion when the infusion tracker is in the second assembled state.

According to an aspect of some embodiments there is further provided a method of controlling and/or authenticating and/or registering an infusion event, the method comprises:

providing an infusion tracker according to the teachings herein, wherein the memory tag stores personal data of a patient and/or data of a care giver, and/or data of a drug to be infused or blood product to be transfused , and/or data of an application activated procedure;

assembling the infusion tracker in the second assembled state so that the infusion tracker encloses a portion of an infusion line configured to deliver a drug to be infused and/or a pharmaceutical fluid and/or a total parenteral nutrition (TPN) fluid there through;

using a processor-controlled device, retrieving personal data of a patient and/or data of a care giver, and/or data of a drug to be infused, and/or activating, in the processor-controlled device, an application activated procedure by commands stored in the memory tag, and

using a computerized system, authenticating the data retrieved from the memory tag and/or registering an infusion event.

Aspects and embodiments of the invention are described in the specification herein below and in the appended claims.

Unless otherwise defined, all technical and scientific terms used herein have the same meaning as commonly understood by one of ordinary skill in the art to which this invention pertains. In case of conflict, the patent specification, including definitions, takes precedence.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

As used herein, the indefinite articles “a” and “an” mean “at least one” or “one or more” unless the context clearly dictates otherwise.

Embodiments of methods and/or devices of the invention may involve performing or completing selected tasks manually, automatically, or a combination thereof. Some embodiments of the invention are implemented with the use of components that comprise hardware, software, firmware or combinations thereof. In some embodiments, some components are general-purpose components such as general purpose computers or oscilloscopes. In some embodiments, some components are dedicated or custom components such as circuits, integrated circuits or software.

For example, in some embodiments, some of an embodiment is implemented as a plurality of software instructions executed by a data processor, for example which is part of a general-purpose or custom computer. In some embodiments, the data processor or computer comprises volatile memory for storing instructions and/or data and/or a non-volatile storage, for example, a magnetic hard-disk and/or removable media, for storing instructions and/or data. In some embodiments, implementation includes a network connection. In some embodiments, implementation includes a user interface, generally comprising one or more of input devices (e.g., allowing input of commands and/or parameters) and output devices (e.g., allowing reporting parameters of operation and results.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIGS. 1A and 1B schematically depict an embodiment of an infusion tracker according to the teachings herein;

FIG. 2A schematically depicts an embodiment of the infusion tracker of FIGS. 1 in a first state being an un-assembled state;

FIG. 2B schematically depicts a cross-section of the infusion tracker of FIGS. 1 in a second, assembled state;

FIGS. 3A-3D schematically depict aspects of a process of activating the application treatment using the infusion tracker of FIGS. 1;

FIGS. 4A-4C schematically depict aspects of a process of verifying the infusion bar code using the infusion tracker tag details of FIGS. 1;

FIGS. 5A and 5B schematically depict an embodiment of an infusion tracker according to the teachings herein;

FIGS. 6A and 6B schematically depict the infusion tracker of FIGS. 5A and 5B in a first assembled state and in a second assembled state, respectively;

FIG. 7A depicts the infusion tracker of FIGS. 5A and 5B in an unassembled state in a semi exploded view;

FIG. 7B depicts the infusion tracker of FIGS. 5A and 5B in an unassembled state when an infusion line is inserted therein;

FIGS. 8A-8C depict schematically aspects of a process of controlling infusion using the infusion tracker of FIGS. 5A and 5B;

FIG. 9 depicts schematically aspects of a process of controlling infusion using the infusion tracker of FIGS. 5A and 5B;

FIG. 10 schematically depict an embodiment of an infusion tracker according to the teachings herein;

FIGS. 11A-11D schematically depict the infusion tracker of FIG. 10 in a first assembled state;

FIGS. 12A-12D schematically depict the infusion tracker of FIG. 10 in a second assembled state;

FIG. 13 schematically depicts a method of controlling and/or authenticating and/or registering an infusion event, using the infusion tracker of FIGS. 1, and

FIG. 14 schematically depicts a method of controlling and/or authenticating and/or registering an infusion event, using the infusion tracker of FIGS. 5 or the infusion tracker of FIG. 10.

### DESCRIPTION OF SOME EMBODIMENTS OF THE INVENTION

The principles, uses and implementations of the teachings herein may be better understood with reference to the accompanying description and figures. Upon perusal of the description and figures present herein, one skilled in the art is able to implement the invention without undue effort or experimentation.

Before explaining at least one embodiment in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

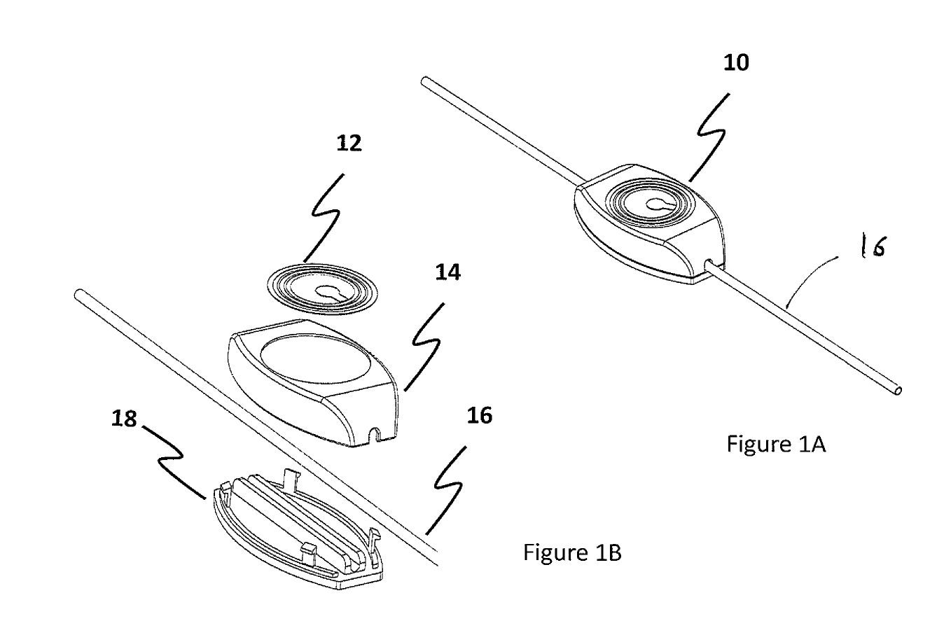

FIGS. 1A and 1B schematically depict an embodiment of an infusion tracker 10 according to an aspect of some embodiments. In FIG. 1A infusion tracker 10 is assembled onto an infusion line 16. In FIG. 1B infusion tracker 10 is depicted in an exploded view. Infusion tracker 10 comprises a cover 14, a base 18 and a memory tag 12 attached to the cover 14.

Infusion tracker 10 is passive, without any integrated control unit. In this option the infusion tracker 10 is only for starting a computerized application by scanning the tag with a smartphone with a dedicated application. The details on the tag form a set of commands to start the application. By starting the application a care giver can scan a barcode of an infusion fluid containing bag, according the application guidance.

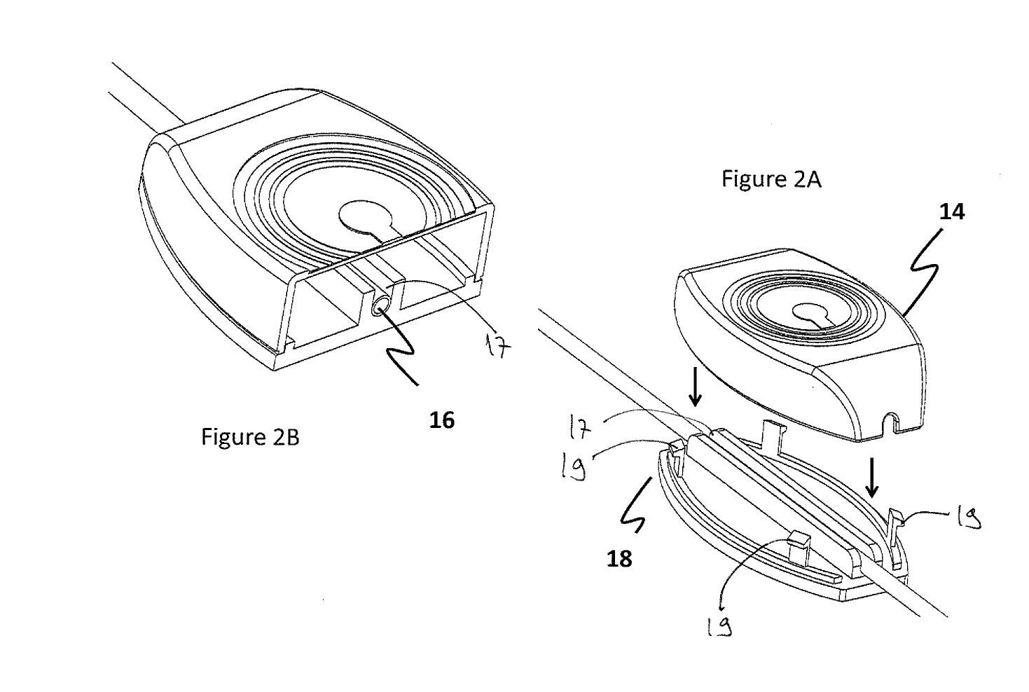

FIG. 2A schematically depicts infusion tracker 10 in a first state being an un-assembled state. FIG. 2B schematically depicts a cross-section of infusion tracker 10 in a second, assembled state. FIG. 2A shows infusion tracker 10 before closing the cover. The infusion line 16 is located in a special notch 17 but remains open for free flow. Infusion tracker 10 does not include a mechanical valve.

The base 18 has 4 snaps 19. By pressing the cover to the base, the snaps 18 enter to a special groove in the cover without any capability to open it after closing—this is a one way movement.

The assembly of infusion tracker 10 may be performed in the pharmacy, after filling an infusion fluid containing bag (not shown) with a medication and labeling. Infusion tracker 10 is assembled on the line as depicted in FIG. 2A. In this option there is no clinical info on the tag. The bag with the medication and with infusion tracker 10 is ready for sending to the patient.

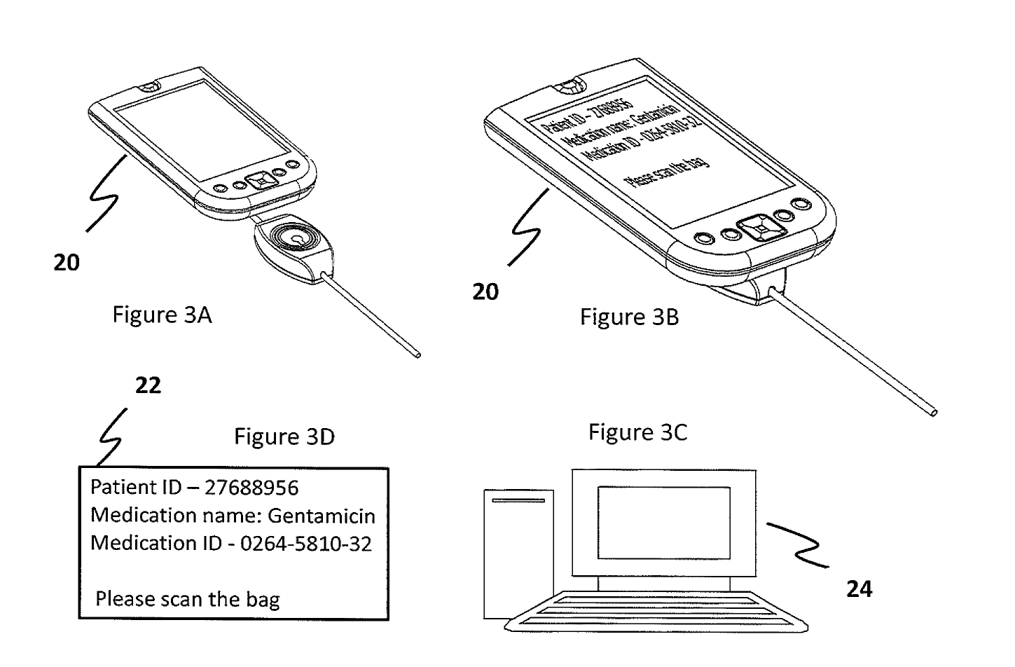

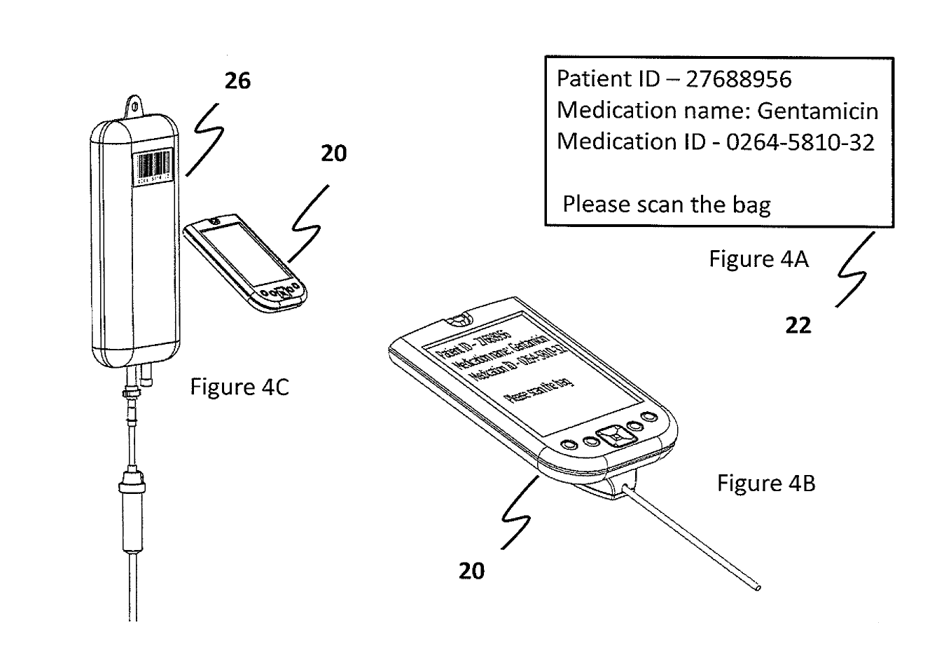

FIGS. 3A-3D and FIGS. 4A-4B depict schematically a process of controlling infusion using Infusion tracker 10. After connecting the bag to the patient by the infusion line 16, a care giver may scan the memory tag 12 on infusion tracker 10 by a smartphone 20 with a dedicated application. By scanning the infusion tracker 10 the application starts working and sends a request to a backend station 24 (FIG. 3C) wirelessly for the treatment details for this patient. The treatment details are received from the back office station 24 and the care giver is asked to scan the bag barcode or NFC tag by a message 22. By scanning the tag the message on the screen (FIGS. 4A, 4B) asks for scanning the barcode bag. The care giver scans the bag barcode (FIG. 4C) and if all the details comparing the physician order are correct, the smartphone sends to the back office confirmation and start monitoring the procedure. According to some embodiments, after completing the treatment the care giver scans the tag again thereby activating sending to the backend an indication that treatment has completed and can throw away the empty set including the infusion tracker. Infusion tracker 10 is a disposable unit.

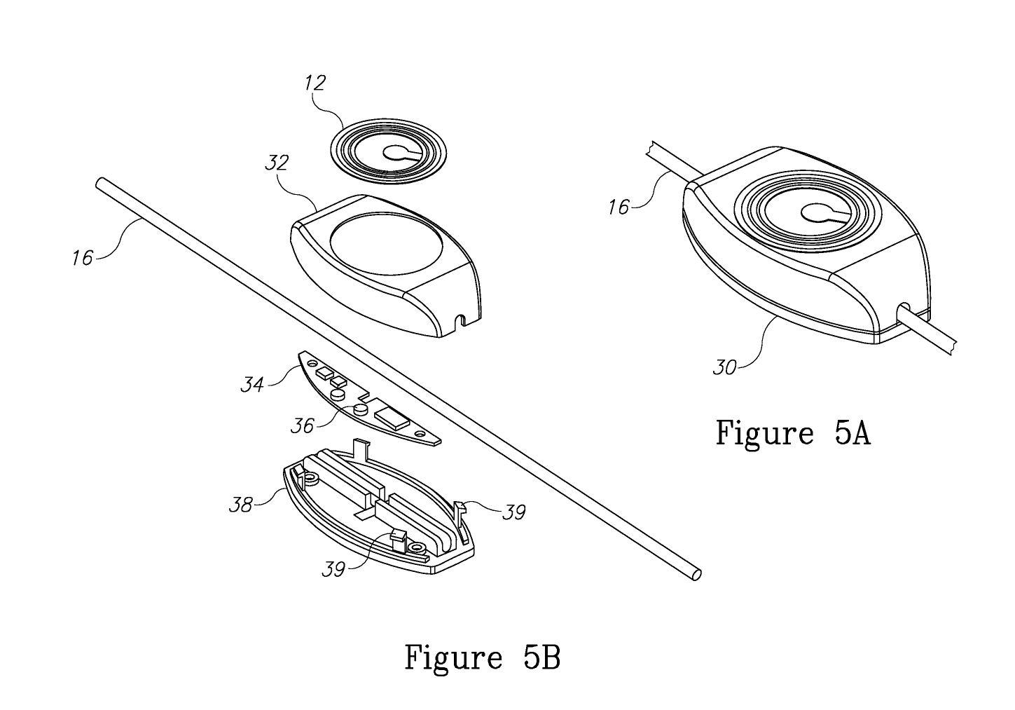

FIGS. 5A and 5B schematically depict an embodiment of an infusion tracker 30 according to an aspect of some embodiments. In FIG. 5A infusion tracker 30 is assembled onto an infusion line 16. In FIG. 5B infusion tracker 30 is depicted in an exploded view. Infusion tracker 30 comprises a cover 32, a base 38 and a memory tag 12 attached to the cover 32. Infusion tracker 30 further comprises a communication board 34 with a processor (not shown). Communication board 34 may comprise a communication channel for communicating with a smartphone or a hand held computerized device. Such a communication may include any RF communication like Bluetooth or others. The communication may include sound signal, e.g. a series of bursts of whistling, or infra-red (IR) burst flickering light. Suitable signals such as the RF signal, the sound or IR signals may connect to the smartphone or to the hand-held device, and activate a dedicated application installed therein. Communication board 34 comprises an activation switch 36, configured to be activated by pressing the cover to the base as is further described below. Base 38 comprises snaps 39 for assembling cover 32 onto base 38.

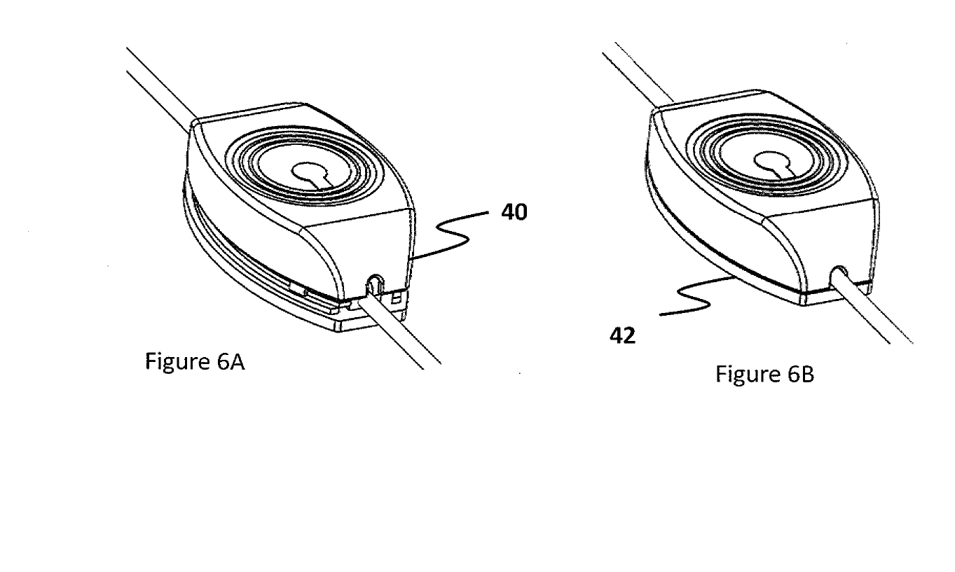

Infusion tracker 30 has two assembled states in which the cover 32 is assembled onto the base 38. FIGS. 6A and 6B schematically depict infusion tracker 30 in the first assembled state 40 and in the second assembled state 42, respectively.

In the first assembled state 40 the snaps 39 are in the first closing position. The PCB 34 is not activated. The first assembled state 40 is performed e.g. by the pharmacy after medication is filled in the infusion bag, the set is ready for sending to the patient for treatment.

In the second assembled state 42 the snaps 39 are in the second closing position. The second assembled state 42 is obtained by pressing the cover 32 onto the base 38 in the first assembled state 40. This movement is one way without any capability to move back to the first assembled state. In this position the PCB 34 is activated by the micro switch 36 and the control unit is activated.

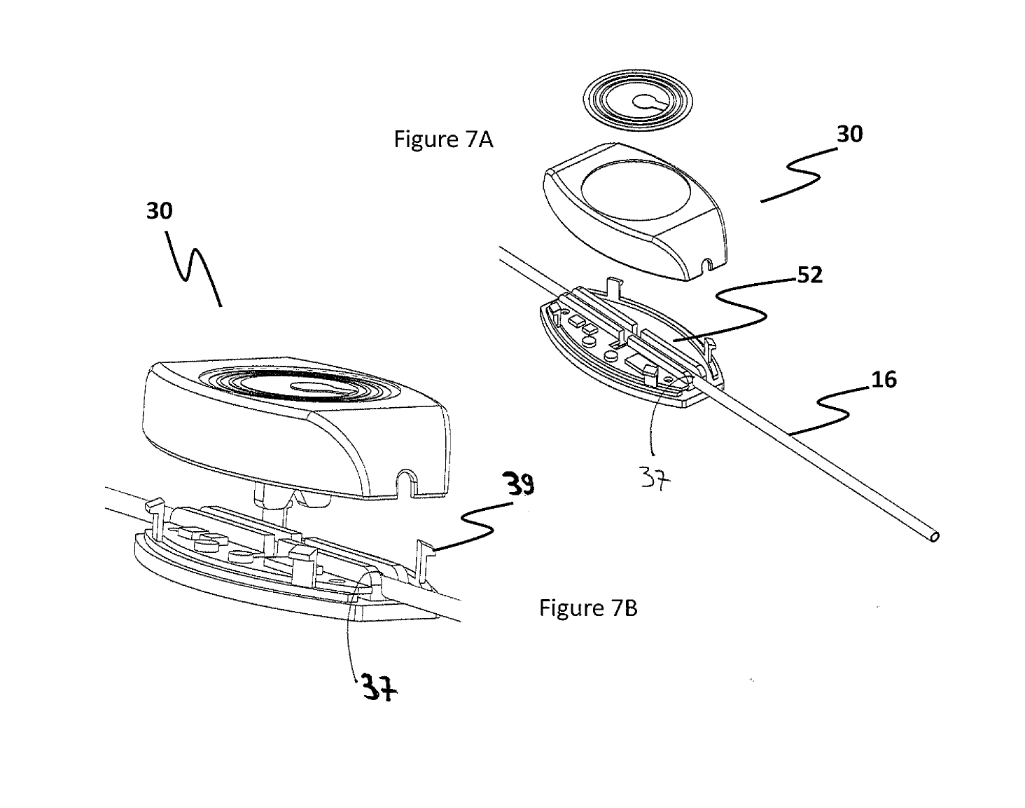

FIG. 7A depicts infusion tracker 30 in an unassembled state in a semi exploded view. FIG. 7B depicts infusion tracker 30 in an unassembled state when an infusion line is inserted into notch 37 in base 38.

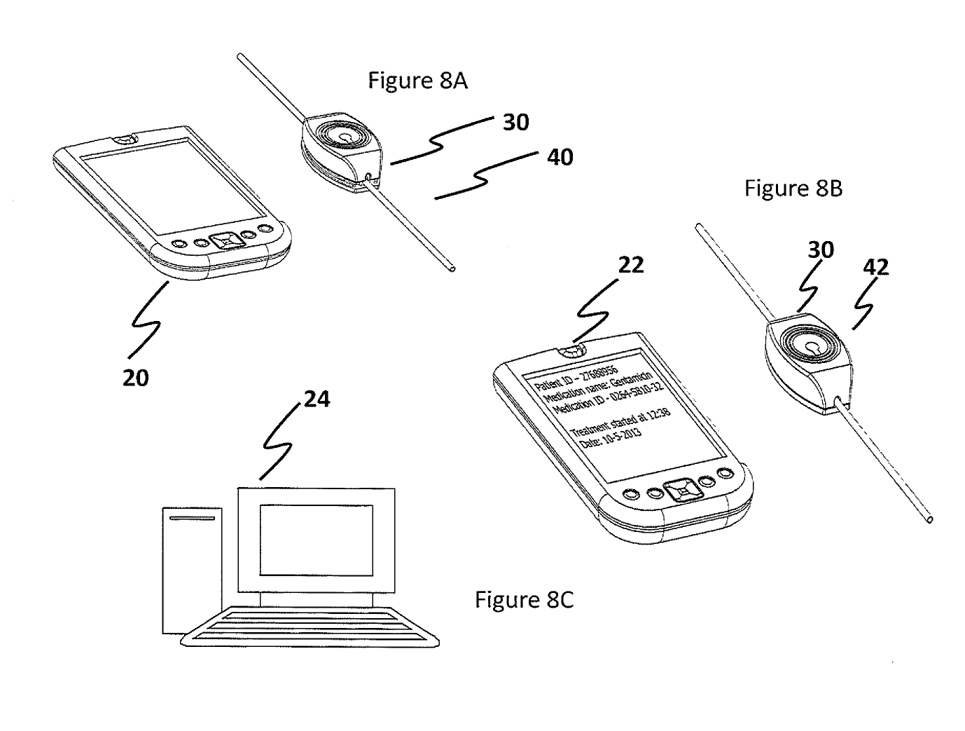

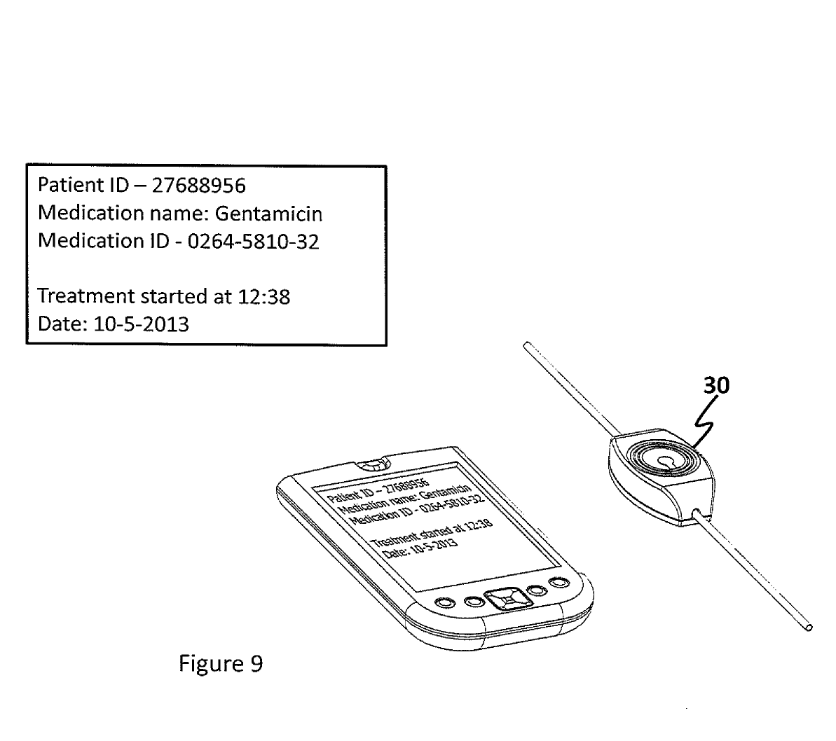

FIGS. 8A-8C and FIG. 9 depict schematically a process of controlling infusion using infusion tracker 30. In FIG. 8A infusion tracker 30 is ready to start, being in the first assembled state and the PCB 34 is not activated yet. In the smartphone or hand-held device 20 a dedicated application is not yet activated.

In FIG. 8B the care giver presses the cover onto the base and the infusion tracker 30 is moved to the second assembled state. The PCB 34 starts sending handshake signal to the smartphone, after handshaking was done a dedicated application starts and patient details are retrieved from the memory tag 12. Patient details are sent to the backend station 24 for treatment information. The application on the hand-held device 20 receives all the treatment details from the backend and the care giver is asked to scan the tag for comparison with the medication ID code and patient ID code (FIG. 9). According to some embodiments, in this option there is no need to scan the medication barcode. If comparing the patient details and medication details indicate an error, backend station 24 may send hazard message to all the client units.

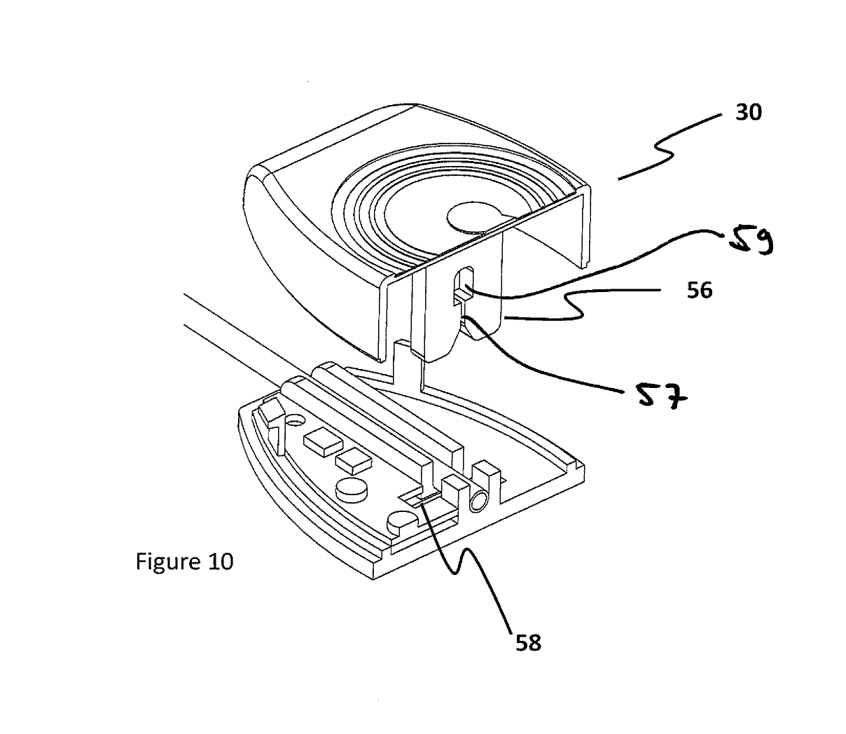

FIG. 10 schematically depicts infusion tracker 30 according to some embodiments. The cover 32 includes a pinch valve 56 having a notch 57 and an opening 59. The notch is positioned on the infusion line according the cover location: in the first assembled state the notch closes the line, and in the second assembled state the notch opens the infusion line and the flow is free to go. Groove 58 in base 38 is configured for pinch valve 56 to be inserted therein when infusion tracker 30 is in the second assembled state.

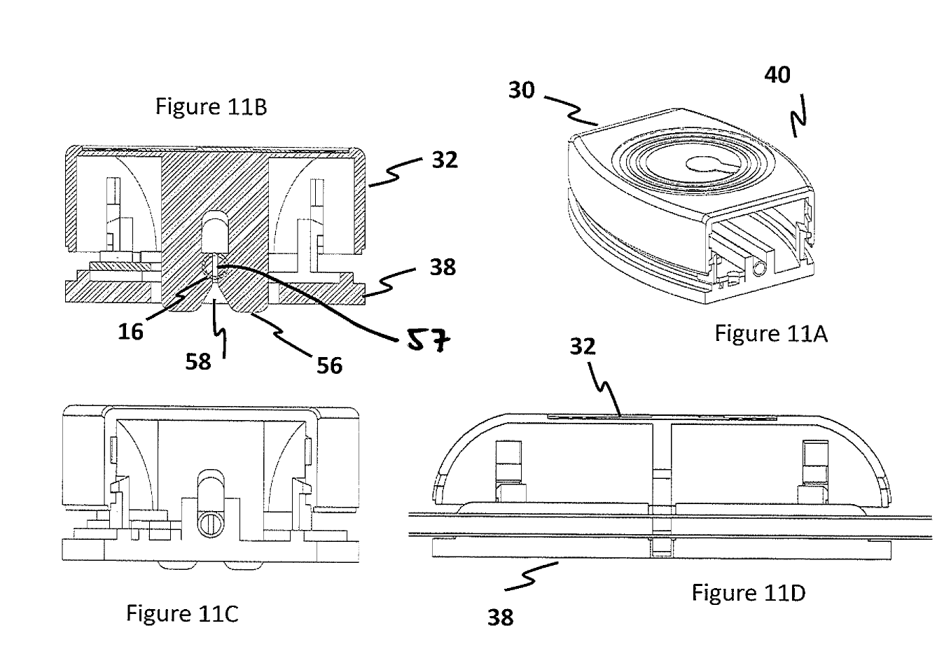

FIGS. 11A-11D schematically depict infusion tracker 30 in the first assembled state 40. The snaps 39 are in the first groove position. The pinch valve 56 is in the closing position, the notch closes the line, no flow is able to go in this position and the flow is blocked. In the first assembled state the set is ready to be sent from the pharmacy to the patient.

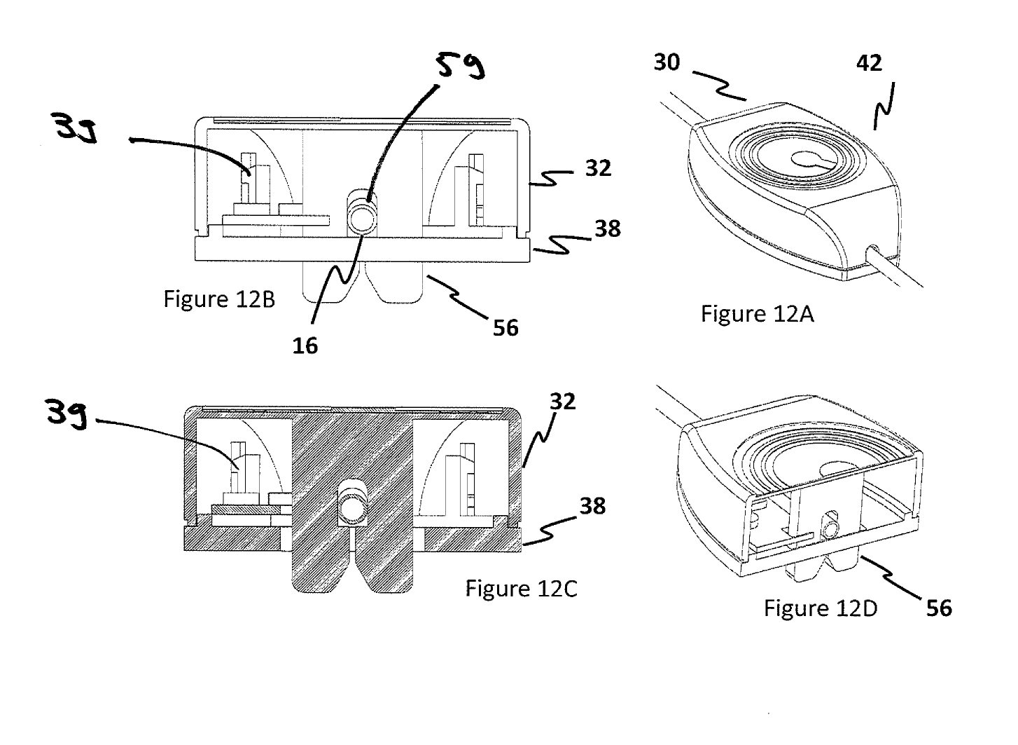

FIGS. 12A-12D schematically depict infusion tracker 30 in the second assembled state 42. The snaps 39 are in the second groove position. The pinch valve 56 is in the opening position, located in the base groove 58. The infusion line goes through the opening 59 in pinch valve 56 and thereby infusion flow is free to go. In the second assembled state the set is ready to use, namely infusion may be infused to the patient.

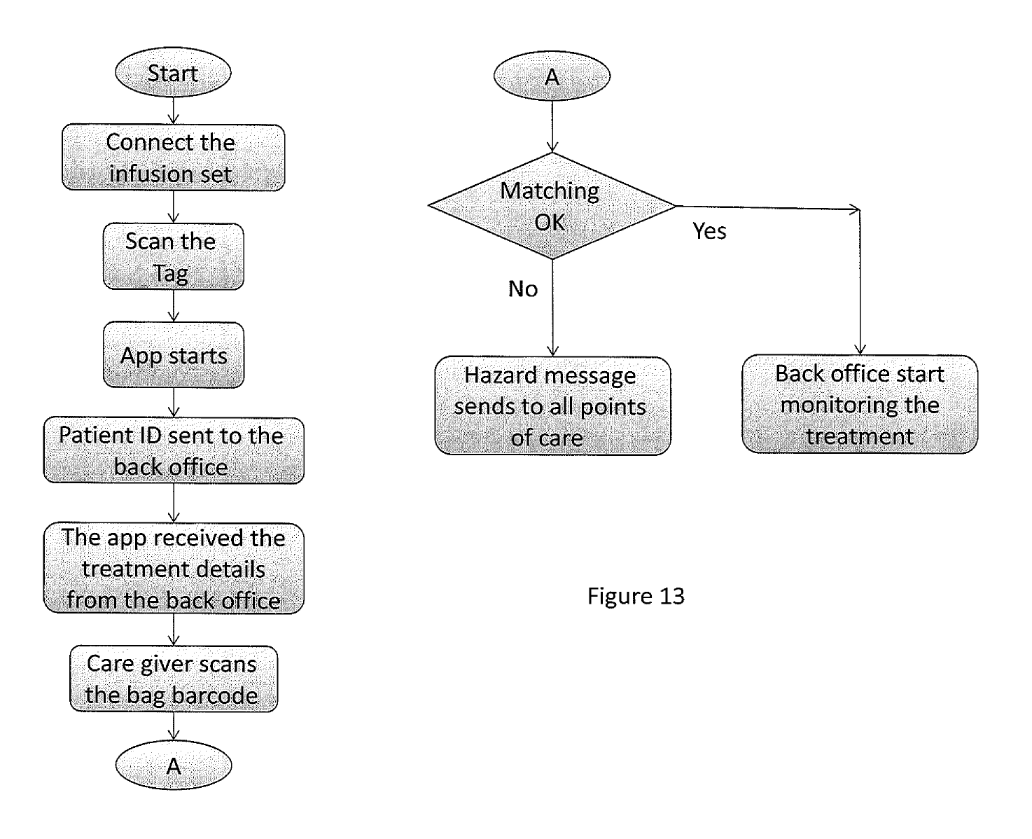

FIG. 13 schematically depicts a method of controlling and/or authenticating and/or registering an infusion event, using infusion tracker 10, according to the teachings herein.

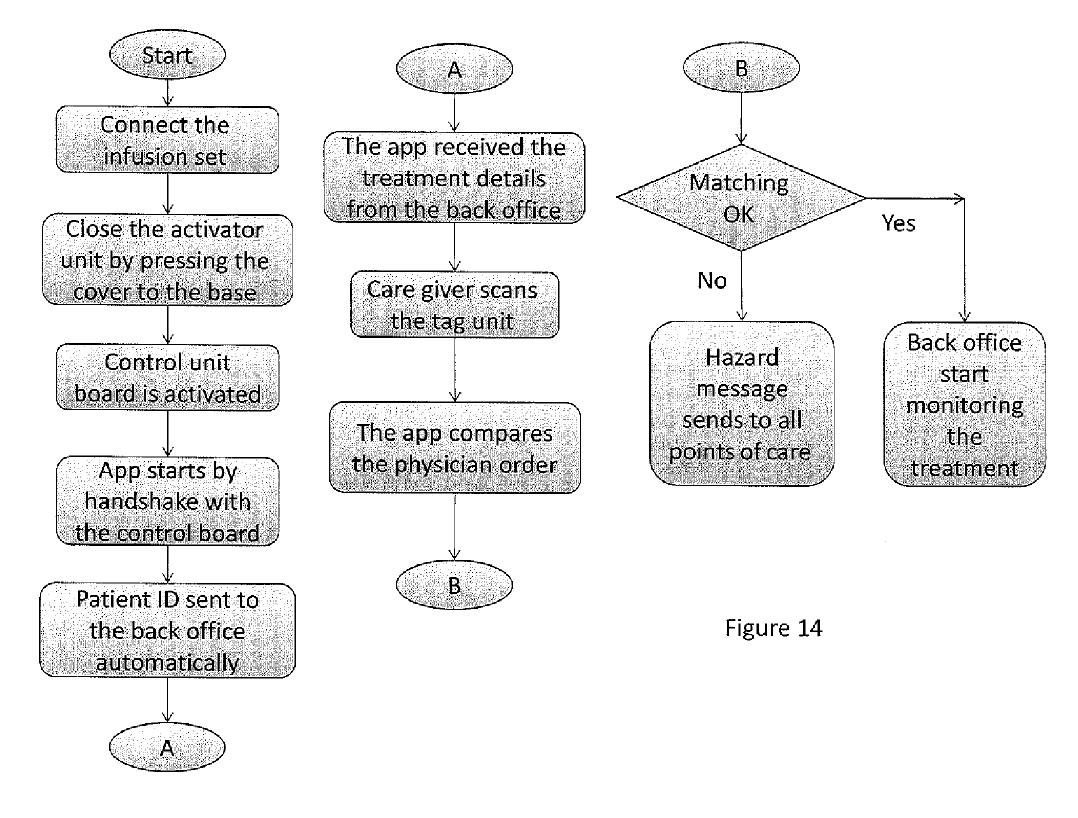

FIG. 14 schematically depicts a method of controlling and/or authenticating and/or registering an infusion event, using infusion tracker 30, according to the teachings herein.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims

Citation or identification of any reference in this application shall not be construed as an admission that such reference is available as prior art to the invention.

Section headings are used herein to ease understanding of the specification and should not be construed as necessarily limiting.

1. An infusion tracker comprising:
a base;
a cover, configured to be assembled onto said base, and
a memory tag, attached to one of said base and said cover and configured to store data and operable to wirelessly release stored data in a format readable by a processor-controlled device or activate an application software in a processor-controlled device,
wherein the infusion tracker has at least a first state, and a second assembled state in to which said cover is assembled onto said base,
and wherein the infusion tracker may be moved from the first state to the second assembled state by manually pressing said cover towards said base, and the infusion tracker may not be moved from said second assembled state to said first state by pressing said cover towards said base nor by releasing a pressure therefrom,
and wherein the infusion tracker, in the second assembled state is further configured to enclose a portion of an infusion line between two ends of said portion, said portion being enclosed substantially between said base and said cover,
and wherein fluid communication substantially exists between the two ends of said portion when the infusion tracker is in said second assembled state.

2. The infusion tracker of claim 1 wherein said second assembled state is a single assembled state, the infusion tracker having no more assembled states in which said cover is assembled to said base.

3. The infusion tracker of claim 1, wherein said first state is an assembled state in which said cover is assembled to said base.

4. The infusion tracker of claim 3 further configured to prevent fluid communication between the two ends of said portion when the infusion tracker is in said first assembled state.

5. The infusion tracker of claim 4 further comprising a valve arranged proximal to said portion when the infusion tracker encloses an infusion line, and configured to press onto the infusion line between said two ends when the infusion tracker is in said first assembled state, thereby preventing fluid communication between the two ends of said portion.

6. The infusion tracker of claim 5 wherein said valve is a pinch valve comprising a plate arranged perpendicular to an infusion line enclosed in the infusion tracker, said plate having a notch in a form of a slit and an opening wider than said notch so that an infusion line enclosed in the infusion tracker is pressed substantially inside said notch when the infusion tracker is in said first assembled state, and an infusion line is substantially disposed in said opening when the infusion tracker is in said second assembled state.

7. The infusion tracker of claim 3 further comprising an electric circuit configured to wirelessly communicate with a processor-controlled device.

8. The infusion tracker of claim 7 wherein said wireless communication includes sound signals or ultrasound signals or light signals or infra-red signals or radio-frequency signals.

9. The infusion tracker of claim 7 wherein said wireless communication is configured to initiate a handshake process establishing a standard protocol communication such as Bluetooth or Wi-Fi, between the infusion tracker and a processor-controlled device.

10. The infusion tracker of claim 7 wherein said electric circuit is activated by assembling the infusion tracker in the second assembled state.

11. The infusion tracker of claim 10 wherein said switch is activated by being depressed between said cover and said base in said second assembled state.

12. The infusion tracker of claim 1, having no electromechanically operated actuator.

13. The infusion tracker of claim 1, wherein said memory tag comprises a Near Field Communication (NFC) tag or RFID tag.

14. The infusion tracker of claim 1, wherein said processor-controlled device is a cell-phone or any hand held device.

15. A method of controlling and/or authenticating and/or registering and/or tracking an infusion event, the method comprises:
providing the infusion tracker of any one of the preceding claims wherein the memory tag stores personal data of a patient and/or data of a care giver, and/or data of a drug to be infused, and/or data of an application activated procedure;
assembling the infusion tracker in the second assembled state so that the infusion tracker encloses a portion of an infusion line configured to deliver a drug to be infused and/or a pharmaceutical fluid and/or a total parenteral nutrition (TPN) fluid and/or blood there through;
using a processor-controlled device, retrieving personal data of a patient and/or data of a care giver, and/or data of a drug to be infused, and/or activating an application activated procedure using a command stored in the memory tag, and
using a computerized system, authenticating the data retrieved from the memory tag and/or registering an infusion event.

16. The method of claim 15 wherein the infusion tracker is according to claim 3 and any one claim dependent thereupon, and the method further comprises, prior to said step of assembling the infusion tracker in the second assembled state, assembling the infusion tracker in the first assembled state so that the infusion tracker encloses a portion of an infusion line configured to deliver a drug to be infused and/or a pharmaceutical fluid and/or a total parenteral nutrition (TPN) fluid there through;

17. The method of claim 16 wherein said step of assembling the infusion tracker in the second assembled state comprises pressing the cover towards the base, thereby moving the infusion tracker from the first assembled state to the second assembled state.

18. The method of claim 17 wherein said pressing step follows said authenticating step.

19. The method of claim 17 wherein the infusion tracker is configured to prevent fluid communication between the two ends of the portion of the infusion line when the infusion tracker is in the first assembled state, thereby allowing infusion only following said pressing step.

20. The method of claim 18 wherein said authenticating step comprises:
using the processor-controlled device to obtain identification data of a pharmaceutical fluid or TPN to be delivered through the infusion line, and
comparing the personal data retrieved from the memory tag to the identification data of the pharmaceutical fluid.
